(19) United States
(12) Patent Application Publication
(10) Pub. No.: US 2006/0149304 A1
(43) Pub. Date: Jul. 6, 2006

(54) Staple needle probe

(76) Inventor: Tien Huang, Arcadia, CA (US)

Correspondence Address: LAW OFFICES OF CLEMENT CHENG, 17220 NEWHOPE STREET #127, FOUNTAIN VALLEY, CA, 92708, US

(21) Appl. No.: 11/356,156

(22) Filed: Feb. 16, 2006

Related U.S. Application Data
(63) Continuation-in-part of application No. 11/026,961, filed on Dec. 30, 2004.

(51) Int. Cl. A61B 17/34

(52) U.S. Cl. 606/189

(57) ABSTRACT

An acupuncture needle has a staple having a pair of points for inserting into skin. The pair of points is connected at a bridge. A bridge and a pair of thermocouple metal members are mounted at opposing ends of an electrical connector when the acupuncture needle is plugged in. The acupuncture needle forms a thermocouple junction for monitoring subdermal patient temperature. Alternatively, a single needle can be used.

This application is a continuation part of copending Acupuncture Needle Thermocouple Clip by same inventor Tien Huang having U.S. Ser. No. 11/026961.

### DISCUSSION OF RELATED ART

Combining a variety of thermocouple metals can create a variety of thermocouples. Usual combinations are called types and identified by a letter such as B, C, E, J, K, N, R S, or T. The different types have different operating ranges and are typically used for different applications. The material of the metals used dictates the different applications. For example, type K has Chromel Alumel metals that generate voltage proportional to a difference in temperature pursuant to a Peltier-Seebeck effect. The voltage is then calibrated and analyzed to output a temperature reading.

Monitoring of patient temperature underneath the skin can give great insight into patient health. Monitoring of patient temperature can produce a diagnosis of insufficient internal heat generation, or under even heat generation. The dynamic balance of a patient thermal generation is also important. Previous attempts at a needle probe relied upon inserting a thick needle housing a thermocouple junction into a patient. Inserting a large diameter needle is particularly painful and disruptive to a patient so that a patient would refuse to be poked in dozens of spots on the skin. When a patient has hot external temperature as measured by a thermal camera, and a lower internal temperature as measured by a probe, this condition is usually medically relevant. Patients may also feel a thermal grill illusion where different portions of their body are at different temperatures, producing discomfort. To diagnose patient thermal problems, a less invasive yet accurate method is required.

### BRIEF DESCRIPTION OF THE DRAWINGS

FIG. 1 is a top view of the first embodiment of the present invention.

FIG. 2 is a side view of the second embodiment of the present invention.

The figures are diagrams and not drawn to scale.

### DETAILED DESCRIPTION OF THE PREFERRED EMBODIMENT

There are a pair of embodiments of for a needle probe, the first embodiment has a staple dual prong, dual needle construction, and the second embodiment has a single prong single needle construction. The staple embodiment is the best mode when combined with an insulated sheath because it allows a thinner needle.

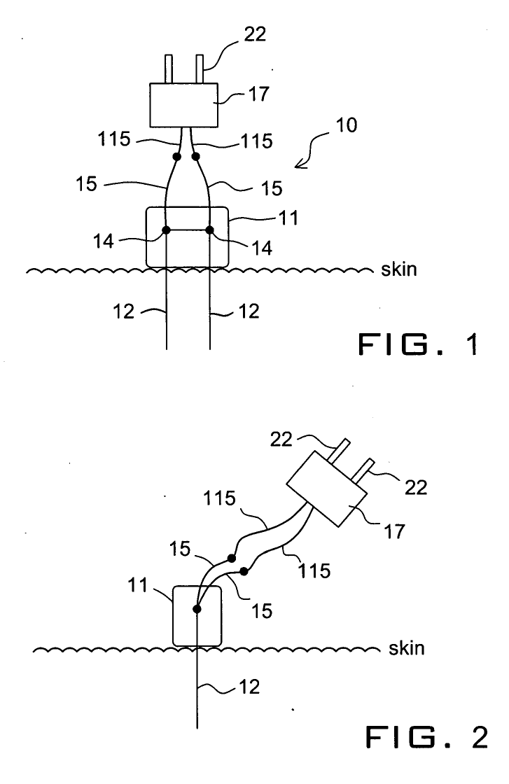

The staple needle probe 10 has a pair of legs 12 extending into the skin of the patient. The legs 12 are shafts of an acupuncture needle 12 having the diameter of the acupuncture needle. In the first embodiment of the invention FIG. 1, the wiring pair 15 is directly connected to the staple 10 at each corner of the staple formed between the legs of the staple 12 and the bridge of the staple. The bridge of the staple connects the legs together. The sheath is both electrically and thermally insulative.

The present device as seen in FIG. 1 is used to monitor patient temperature. The staple needle probe 10 is inserted into skin for treatment of a patient. A staple needle 10 is formed of a pair of acupuncture needle shafts 12 typically made of metal and can vary in width between the needles 12. The staple needle 10 is at a thermocouple junction where a pair of wire leads 15 or other electrical connection 15 is connected to the needle 10. A sheath or plastic housing 111 preferably covers the needle 10 and the junction area 14 at which the staple and wire connect.

The wire leads 15 can then be plugged into a plug 17. The staple needle probe 10 may have a sheath 11 that exposes a length of acupuncture needle 12. The length exposed can be indicated on the housing or sheath 11. The housing 11 preferably forms a grip for convenient manual use. The housing is preferably light material and fully encapsulating the junction 14. The depth of the insertion is uniformly measured by the housing 11 that limits the insertion of the acupuncture needle shaft 12 to a uniform depth. The bottom portion of the housing 11 abuts the skin when fully inserted. The staple dimensions can be sized according to application.

The different material types and thermocouple types are defined in the wire lead 15 materials. The wire lead materials can have a varying number of metals including metals of the B, C, E, J, K, N, R S, or T type. The wire leads can be selected to maintain acceptable sensitivity and range. However, the Standard K type thermocouple is sufficient for most purposes. A usual K type thermocouple can be implemented with a pair of different wire lead 15 materials.

As previously disclosed, the wire leads connect to the acupuncture needle shaft 12. The electrical wire mounted to the acupuncture needle carries a voltage to a data analyzer and data logger for signal processing.

A staple needle probe, or single needle probe can have a variety of electrical connections, including a clip, solder, plug type configuration. A variety of clips and their corresponding thermocouple type electrical wire members can thus be selected depending upon the thermocouple type desired.

The acupuncture needle can be made of a variety of metals including stainless steel, gold, and silver. The acupuncture needle forms the thermocouple junction of the thermocouple sensor and becomes a part of the sensor.

The needle must comply with federal and state law for material and sterility. A staple needle 10 can be connected to the wire leads 15 by an electrical connector that allows modular connection. The plug 17 has plug prongs 22 that connect into the socket of the data logger also called the data analyzer that converts the voltage between the plug prongs 22 into a temperature reading. The wire leads 15 are insulated wires that can be braided or otherwise gathered together so that a user perceives handling of a single cable extending between the plug 17 and the sheath 11.

The acupuncture needle 10 formed in the shape of a staple can be replaced. The connector can eject the used acupuncture needle 10 and retain the remainder of the device including the wiring 15 connecting to plug 17. In this case, the thermocouple materials can be formed within the wiring 15, or plug 17, allowing the wiring 15 to be made of the same material. The electrical connection 29 electrically connects the wiring 15 to the staple 10 when engaged. The electrical connection 29 can be a copper electrical connector formed to make the electrical connection. When the thermocouple materials are mounted within the electrical connector, the electrical connector 29 can be made of a pair of thermocouple materials.

Alternatively, in a direct connection that is the electrical connection configuration without a connector where the wires 15 are directly connected to the acupuncture needle staple 10, the replacement part would be the staple 10, sheath 11, wires 15, and plug 17. For typical use on a patient, the connection may be a permanent connection that is either soldered or otherwise electrically connected.

The second embodiment that is not the best mode is a single needle probe. The single needle probe can be constructed in the same fashion as the staple needled probe. The needle 12 can be inserted into skin and first attached to a pair of wires 15 at a junction. The wires may be a thermal couple pair having a pair of thermocouple materials. To conserve thermocouple material, a length of thermocouple material in the wires 15 connect to ordinary wires such as a pair of copper wires 115. The standard wiring 115 is connected to terminals on a plug 17 and thus electrically connected to plug prongs 22. The plug prongs 22 fit into a data logger and thus should be standard sized for available data loggers. The embodiments based on the description provided can be constructed inexpensively with virtually no experimentation and using standard currently commercially available parts.
10 Staple Needle
11 Sheath
12 Acupuncture Needle Shaft
14 Staple Wire Connection
15 Wire Lead
17 Plug
22 Plug Prong
29 Electrical Connection

1. An acupuncture needle probe comprising:
a. a staple having two legs of acupuncture needles each having a point for inserting into skin, wherein the pair of legs form leg upper ends connected at a bridge;
b. wire leads connected to the upper ends of the legs; wherein the acupuncture needle forms a thermocouple junction.

2. The acupuncture needle probe of claim 1, wherein the wires leading to the upper ends of the legs connected to the junction are made of dissimilar thermocouple materials and the legs are made of the same material.

3. The acupuncture needle probe of claim 1, wherein the wires leading to the upper ends of the legs connected to the junction are made of the same material and the legs are made of dissimilar thermocouple materials.

4. The acupuncture needle probe of claim 1, wherein the wires leading to the upper ends of the legs connected to the junction are made of dissimilar thermocouple materials and the legs are made of dissimilar thermocouple materials.

5. An acupuncture needle comprising:
a. a staple having a pair of points for inserting into skin, wherein the pair of points are acupuncture needles and are connected at a bridge;
b. an electrical connector for attaching to the bridge;
c. a pair of thermocouple metal members mounted at opposing ends of the bridge;
d. wherein the clip and a pair of grasping ends are shaped to grasp an acupuncture needle, whereby the acupuncture needle forms a thermocouple junction.

6. The acupuncture needle of claim 5, wherein the wires leading to the upper ends of the legs connected to the junction are made of dissimilar thermocouple materials and the legs are made of the same material.

7. The acupuncture needle of claim 5, wherein the wires leading to the upper ends of the legs connected to the junction are made of the same material and the legs are made of dissimilar thermocouple materials.

8. The acupuncture needle of claim 5, wherein the wires leading to the upper ends of the legs connected to the junction are made of dissimilar thermocouple materials and the legs are made of dissimilar thermocouple materials.

9. An acupuncture needle temperature monitoring device comprising:
a. a single acupuncture needle;
b. a pair of thermocouple metal members mounted to the single acupuncture needle, wherein the acupuncture needle forms a thermocouple junction;
c. an electrical conductor electrically conducting the pair of thermocouple metal members to output a voltage.

10. The acupuncture needle of claim 9, wherein the needle is formed as a staple with a pair of points on a pair of legs connected by a bridge, wherein wires lead to the upper ends of the legs.

11. The acupuncture needle of claim 10, wherein the wires leading to the upper ends of the legs connected to the junction are made of dissimilar thermocouple materials and the legs are made of the same material.

12. The acupuncture needle of claim 10, wherein the wires leading to the upper ends of the legs connected to the junction are made of the same material and the legs are made of dissimilar thermocouple materials.

13. The acupuncture needle of claim 10, wherein the wires leading to the upper ends of the legs connected to the junction are made of dissimilar thermocouple materials and the legs are made of dissimilar thermocouple materials.
